(19) United States
(12) Patent Application Publication
(10) Pub. No.: US 2026/0108232 A1
(43) Pub. Date: Apr. 23, 2026

(54) ULTRASONIC DIAGNOSTIC APPARATUS, MEDICAL IMAGE PROCESSING APPARATUS, AND METHOD

(71) Applicant: CANON MEDICAL SYSTEMS CORPORATION, Tochigi (JP)

(72) Inventors: Hiroki TAKAHASHI, Nasushiobara (JP); Takatoshi OKUMURA, Yaita (JP); Sadanori TOMIHA, Nasushiobara (JP)

(73) Assignee: CANON MEDICAL SYSTEMS CORPORATION, Tochigi (JP)

(21) Appl. No.: 19/365,878

(22) Filed: Oct. 22, 2025

(30) Foreign Application Priority Data
Oct. 22, 2024 (JP) 2024-185833

(51) Int. Cl. A61B 8/00; A61F 7/00; G01K 3/14; G01K 11/24

(52) U.S. Cl. CPC A61B 8/5223; A61B 8/5261; A61F 7/00; G01K 3/14; G01K 11/24

(57) ABSTRACT

An ultrasonic diagnostic apparatus of an embodiment includes processing circuitry. The processing circuitry acquires distribution of tissue properties within a subject. The processing circuitry changes distribution of temperature within the subject. The processing circuitry acquires temperature-change distribution within the subject with the distribution of temperature that has been changed. The processing circuitry generates a thermophysical properties distribution within the subject based on a plurality of distributions of tissue properties acquired in a plurality of states in which the distribution of temperature has been changed and the temperature-change distribution within the subject corresponding to the plurality of states.

### CROSS-REFERENCE TO RELATED APPLICATIONS

This application is based upon and claims the benefit of priority from Japanese Patent Application No. 2024-185833, filed on October 22, 2024; the entire contents of which are incorporated herein by reference.

### FIELD

Embodiments described herein relate generally to an ultrasonic diagnostic apparatus, a medical image processing apparatus, and a method.

### BACKGROUND

Conventionally, it has been known that the speed of sound in biological tissue is related to the temperature of that tissue. For example, in a case of soft tissue, the speed of sound increases as the temperature rises, but in a case of fatty tissue, the speed of sound decreases as the temperature rises. Furthermore, techniques for determining tissue characterization by utilizing differences in the change in the speed of sound accompanying such temperature changes (thermophysical properties) are also known.

To utilize the differences in the change in speed of sound described above, high-precision sound speed measurement methods are essential, and in recent years, technologies for imaging local sound speed within living tissue have advanced, such as sound speed distribution reconstruction using artificial intelligence (AI) and ultrasonic computed tomography (CT).

### BRIEF DESCRIPTION OF THE DRAWINGS

FIG. 1 is a block diagram illustrating an example of a configuration of an ultrasonic diagnostic apparatus according to a first embodiment;

FIG. 2 is a flowchart illustrating a processing procedure of the ultrasonic diagnostic apparatus according to the first embodiment;

FIG. 3 is a diagram for explaining an example of sound speed distribution according to the first embodiment;

FIG. 4 is a diagram for explaining an example of temperature-change distribution according to the first embodiment;

FIG. 5 is a diagram for explaining an example of a thermophysical properties distribution according to the first embodiment;

FIG. 6 is a flowchart illustrating a processing procedure of the ultrasonic diagnostic apparatus according to a second embodiment; and

FIG. 7 is a block diagram illustrating an example of a configuration of a medical image processing apparatus according to another embodiment.

### DETAILED DESCRIPTION

An ultrasonic diagnostic apparatus according to an embodiment includes processing circuitry. The processing circuitry is configured to acquire distribution of tissue properties within a subject. The processing circuitry is configured to change distribution of temperature within the subject. The processing circuitry is configured to acquire temperature-change distribution within the subject with the distribution of temperature that has been changed. The processing circuitry is configured to generate the thermophysical properties distribution within the subject based on a plurality of distributions of tissue properties acquired in a plurality of states in which the distribution of temperature has been changed and the temperature-change distribution within the subject corresponding to the plurality of states.

Hereinafter, embodiments of a medical image processing apparatus, an ultrasonic diagnostic apparatus, a method, and a program according to the present application will be described in detail with reference to the attached drawings. Note that the medical image processing apparatus, the ultrasonic diagnostic apparatus, the method, and the program according to the present application are not limited to the following embodiments.

### First Embodiment

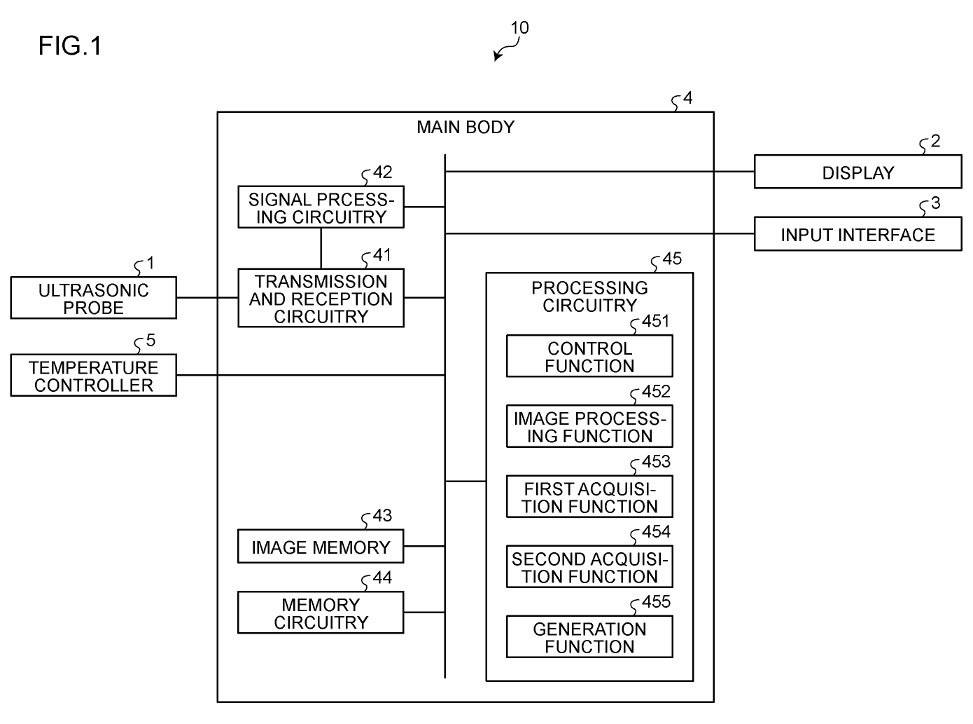

FIG. 1 is a block diagram illustrating an example of a configuration of an ultrasonic diagnostic apparatus 10 according to the first embodiment. As illustrated in FIG. 1, the ultrasonic diagnostic apparatus 10 according to the present embodiment includes an ultrasonic probe 1, a display 2, an input interface 3, and a main body 4, and the ultrasonic probe 1, the display 2, and the input interface 3 are communicably connected to the main body 4. In addition, the ultrasonic diagnostic apparatus 10 is connected to a temperature controller 5 as illustrated in FIG. 1.

The temperature controller 5 changes the distribution of temperature within the subject. For example, the temperature controller 5 is any one of a means for heating by ultrasonic acoustic energy (for example, an ultrasonic heater), a means for heating by electromagnetic interference (for example, an electromagnetic wave heater), a means for heating by optical interference (for example, an infrared heater), and a means for heating by heat transfer from a heat source (for example, an electric current heater), and heats the subject under control by the ultrasonic diagnostic apparatus 10. Here, the temperature controller 5 may be formed in a ring shape so as to surround parts of the subject whose temperature is to be controlled.

In a case where the temperature controller 5 is an ultrasonic heater, the temperature controller 5 includes, for example, an ultrasonic probe and a transmission circuit, and heats the subject using control based on the heating technology known as high-intensity focused ultrasound (HIFU).

In addition, in a case where the temperature controller 5 is an electromagnetic wave heater, the temperature controller 5 includes, for example, an oscillator that emits microwaves or the like, and heats the subject by emitting microwaves from the oscillator. In addition, in a case where the temperature controller 5 is an electric current heater, the temperature controller 5 includes, for example, electrodes facing each other to sandwich the subject, and heats the subject by passing an electric current through the electrodes.

Note that each of the examples of the temperature controller 5 described above is merely an example, and may be a case where another device is used. For example, an infrared heater such as a halogen heater or a carbon heater may be used.

In addition, the temperature controller 5 may be controlled by another device other than the ultrasonic diagnostic apparatus 10. Furthermore, the temperature controller 5 does not necessarily only change the temperature from outside the subject's body but may also change the temperature from within the subject's body. In such cases, for example, the dialyzer is used as the temperature controller 5, and the temperature within the subject is changed by returning blood whose temperature has been adjusted by the dialyzer back into the body. Additionally, the temperature controller 5 may be configured not only to heat but also to cool the inside of the subject. In such cases, for example, the temperature controller 5 lowers the temperature of the inside of the subject by vapor cooling.

The ultrasonic probe 1 includes a plurality of piezoelectric transducer elements that generates ultrasonic waves based on a driving signal supplied by transmission and reception circuitry 41. In addition, the ultrasonic probe 1 receives a reflected wave from the subject to transform the wave to an electrical signal. Furthermore, the ultrasonic probe 1 includes a matching layer provided on the piezoelectric transducer element and a backing material or the like that prevents the propagation of ultrasonic waves rearward from the piezoelectric transducer element. Note that the ultrasonic probe 1 is detachably connected to the main body 4.

When ultrasonic waves are transmitted from the ultrasonic probe 1 to the subject, the transmitted ultrasonic waves are successively reflected at a discontinuity in acoustic impedance within the subject's body tissues. These reflected waves are then received by the plurality of piezoelectric transducer elements included within the ultrasonic probe 1 as reflected wave signals. The amplitude of the received reflected wave signal depends on the difference in acoustic impedance at the discontinuity where the ultrasonic wave is reflected. Furthermore, when the transmitted ultrasonic pulse is reflected off surfaces such as moving blood flow or the heart wall, the reflected wave signal undergoes frequency shift due to the Doppler effect, depending on the velocity component of the moving object relative to the direction of ultrasonic transmission.

The ultrasonic probe 1 may be a one-dimensional ultrasonic probe with a plurality of piezoelectric transducer elements arranged in a single row, or may be an ultrasonic probe that mechanically oscillates the plurality of piezoelectric transducer elements of the one-dimensional ultrasonic probe, or may be a two-dimensional ultrasonic probe with the plurality of piezoelectric transducer elements arranged in a grid pattern in two dimensions.

Here, the ultrasonic probe 1 of the ultrasonic diagnostic apparatus 10 according to the present embodiment has a configuration that enables ultrasonic CT scanning. Specifically, the ultrasonic probe 1 is configured such that the plurality of piezoelectric transducer elements is each positioned opposite one another across the subject, enabling a plurality of scans to be performed at different angles relative to the subject. For example, the ultrasonic probe 1 may have the plurality of piezoelectric transducer elements arranged in a ring shape, or may be configured such that the plurality of opposing piezoelectric transducer elements rotates around the subject using a motor or similar device as the drive source. Note that the above description is merely an example, and the ultrasonic probe 1 may have any configuration that enables ultrasonic CT scanning.

The display 2 displays a graphical user interface (GUI) for an operator of the ultrasonic diagnostic apparatus 10 to input various setting requests using the input interface 3, an ultrasonic image generated in the main body 4, or the like. In addition, the display 2 displays various messages and display information in order to inform the operator of a processing status and processing results of the main body 4. Furthermore, the display 2 includes a speaker and can also output audio.

The input interface 3 is operated to perform setting a predetermined position (for example, the position of a region of interest (ROI)) or the like, and is implemented by a trackball, a switch button, a mouse, a keyboard, a touchpad that performs an input operation by touching an operating surface, a touch monitor integrating a display screen and touchpad, a non-contact input circuit using an optical sensor, an audio input circuit, and the like. The input interface 3 is connected to processing circuitry 45 to be described later, converts an input operation accepted from the operator into an electrical signal, and outputs the electrical signal to the processing circuitry 45. Note that in the present specification, the input interface 3 is not limited to the one having a physical operation part such as a mouse, keyboard, etc. For example, examples of the input interface include a processing circuit of an electrical signal that receives an electrical signal corresponding to an input operation from an external input device mounted separately from the device and outputs the electrical signal to the processing circuitry 45.

The main body 4 is an apparatus that generates ultrasonic images based on the reflected wave signal received by the ultrasonic probe 1, and includes, as illustrated in FIG. 1, a transmission and reception circuitry 41, signal processing circuitry 42, an image memory 43, memory circuitry 44, and processing circuitry 45. The transmission and reception circuitry 41, the signal processing circuitry 42, the image memory 43, the memory circuitry 44, and the processing circuitry 45 are connected to be capable of mutual communication. In the ultrasonic diagnostic apparatus 10 illustrated in FIG. 1, each processing function is stored in the memory circuitry 44 in a program form executable by a computer. The transmission and reception circuitry 41, the signal processing circuitry 42, and the processing circuitry 45 are processors implementing functions corresponding to each program by reading out a program from the memory circuitry 44 and executing the program. In other words, each circuitry in a state where each program was read out has a function corresponding to the program read out.

The transmission and reception circuitry 41 includes a pulse generator, a transmission delay unit, a pulser, or the like, and supplies the driving signal to the ultrasonic probe 1. The pulse generator repeatedly generates a rate pulse for generating transmission ultrasonic waves at a predetermined rate frequency. The transmission delay unit focuses the ultrasonic waves generated by the ultrasonic probe 1 into a beam and applies the delay time required for each piezoelectric transducer element to determine the transmission directionality to each rate pulse generated by the pulse generator. The pulser applies the driving signal (driving pulse) to the ultrasonic probe 1 at timing based on the rate pulse. That is, the transmission delay unit changes the delay time applied to each rate pulse to arbitrarily adjust the transmission direction of the ultrasonic waves transmitted from the piezoelectric transducer element surface

Furthermore, the transmission and reception circuitry 41 has a function capable of instantly changing the transmission frequency, transmission driving voltage, or the like in order to execute a predetermined scan sequence based on an instruction of the processing circuitry 45 to be described later. In particular, changing the transmission driving voltage is achieved by a linear amplifier-type oscillation circuit capable of instantly switching its value, or by a mechanism that electrically switches between a plurality of power supply units.

Furthermore, the transmission and reception circuitry 41 includes a preamplifier, an analog-to-digital (A/D) converter, a reception delay unit, an adder, and the like, and performs various processing operations on the reflected wave signal received by the ultrasonic probe 1 to generate reflected wave data. The preamplifier amplifies the reflected wave signal for each channel. The A/D converter converts the amplified reflected wave signal into a digital signal. The reception delay unit provides a delay time required to determine the reception directionality. The adder performs addition processing of the reflected wave signal processed by the reception delay unit to generate reflected wave data. The addition processing by the adder emphasizes the reflected component from the direction corresponding to the reception directionality of the reflected wave signal, and forms a comprehensive beam for ultrasonic transmission and reception based on the reception directionality and the transmission directionality.

The signal processing circuitry 42 performs, for example, logarithmic amplification, envelope detection processing or the like on the reflected wave data received from the transmission and reception circuitry 41, and generates data (B-mode data) in which the signal intensity at each sample point is represented as brightness. The B-mode data generated by the signal processing circuitry 42 is output to the processing circuitry 45.

Furthermore, the signal processing circuitry 42 generates, for example, data (Doppler data) obtained by extracting motion information based on the Doppler effect of the moving object at each sample point within the scanning area from the reflected wave data received from the transmission and reception circuitry 41. Specifically, the signal processing circuitry 42 performs frequency analysis of velocity information from the reflected wave data, extracts blood flow, tissue, and contrast agent echo components based on the Doppler effect, and generates data (Doppler data) obtained by extracting moving object information such as average velocity, dispersion, and power for multiple points. Here, the moving object refers to, for example, blood flow, tissues such as the heart wall, or contrast agents. The motion information (blood flow information) obtained by the signal processing circuitry 42 is sent to the processing circuitry 45 and displayed in color on the display 2 as an average velocity image, a dispersion image, a power image, or a combination image thereof.

The image memory 43 is a memory storing image data for display generated by the processing circuitry 45. In addition, the image memory 43 can also store data generated by the signal processing circuitry 42. The B-mode data and Doppler data that the image memory 43 stores can be called by the operator after diagnosis, for example, and are processed via the processing circuitry 45 to become ultrasonic image for display.

The memory circuitry 44 stores a control program for performing ultrasonic transmission and reception, image processing, and display processing, diagnosis information (for example, patient ID, physician's findings), and various types of data such as diagnostic protocols, and various body marks. In addition, the memory circuitry 44 stores processing results of the transmission and reception circuitry 41, the signal processing circuitry 42, and the processing circuitry 45. Furthermore, the memory circuitry 44 is also used for storing image data stored in the image memory 43, as necessary. Furthermore, the data stored by the memory circuitry 44 can be transferred to an external device via an interface (not illustrated). Furthermore, the memory circuitry 44 stores temperature-change distribution, and the detail of the temperature-change distribution will be described later. The memory circuitry 44 is an example of the storage circuitry.

The processing circuitry 45 controls the entire processing of the ultrasonic diagnostic apparatus 10. Specifically, the processing circuitry 45 controls processing of the transmission and reception circuitry 41 and the signal processing circuitry 42 based on various setting requests input by the operator via the input interface 3, and various control programs and various types of data read out from the memory circuitry 44. In addition, the processing circuitry 45 controls the ultrasonic image for display stored in the image memory 43 to be displayed in the display 2.

The processing circuitry 45 executes, as illustrated in FIG. 1, a control function 451, an image processing function 452, a first acquisition function 453, a second acquisition function 454, and a generation function 455. Here, the processing circuitry 45 is an example of the processing circuitry.

The control function 451 controls processing of the transmission and reception circuitry 41 and the signal processing circuitry 42 based on various setting requests input by the operator via the input interface 3, and various control programs and various types of data read out from the memory circuitry 44. For example, the control function 451 executes control related to the ultrasonic CT scanning. In addition, the control function 451 controls the ultrasonic image, various kinds of information (for example, thermophysical properties distribution, or the like) to be displayed on the display 2. In addition, the control function 451 controls the temperature controller 5 to change the temperature within the subject.

The image processing function 452 generates an ultrasonic image from data generated by the signal processing circuitry 42. That is, the image processing function 452 generates an ultrasonic image in which the intensity of the reflected wave is represented as brightness from the B-mode data generated by the signal processing circuitry 42. In addition, the image processing function 452 generates an ultrasonic image representing the moving object information (blood flow information and tissue movement information) from the Doppler data generated by the signal processing circuitry 42. The ultrasonic image based on the Doppler data is velocity image data, dispersion image data, power image data, or a combination image data thereof.

Here, the image processing function 452 generally converts (scan-converts) the scan line signal sequence of the ultrasonic scan into a scan line signal sequence in a video format, such as that used in televisions or the like, thereby generating an ultrasonic image for display. Specifically, the image processing function 452 generates an ultrasonic image for display by performing coordinate transformation according to the scanning pattern of the ultrasonic waves from the ultrasonic probe 1. In addition, the image processing function 452 performs various types of image processing in addition to scan conversion, for example, image processing that regenerates an average luminance image using multiple image frames after scan conversion (smoothing processing), and image processing using a differential filter within the image (edge enhancement processing). In addition, the image processing function 452 combines the ultrasonic image with character information, scales, body marks, and the like of various parameters.

That is, the B-mode data and Doppler data are ultrasonic image data prior to scan conversion processing, while the data generated by the image processing function 452 is ultrasonic image data for display after scan conversion processing. Furthermore, in a case where the signal processing circuitry 42 generates three-dimensional data (three-dimensional B-mode data and three-dimensional Doppler data), the image processing function 452 generates volume data by performing coordinate transformation according to the scanning pattern of the ultrasonic waves from the ultrasonic probe 1. Then, the image processing function 452 applies various kinds of rendering processing on the volume data to generate two-dimensional image data for display.

The first acquisition function 453 acquires distribution of tissue properties within the subject. Specifically, the first acquisition function 453 acquires local distribution of tissue properties whose values vary accompanying temperature changes. That is, the first acquisition function 453 acquires tissue property values at each position within the three-dimensional space of the subject. Here, the first acquisition function 453 acquires distribution of tissue properties of each of a plurality of states that changes the temperature within the subject. The above-described tissue properties include, for example, sound speed, computed tomography (CT) values, and nuclear magnetic resonance (NMR) parameters, and the first acquisition function 453 in the ultrasonic diagnostic apparatus 10 according to the first embodiment acquires the local distribution of sound speed. That is, the first acquisition function 453 acquires sound speed distribution indicating sound speed at each position within the subject.

The second acquisition function 454 acquires temperature-change distribution within the subject. Specifically, the second acquisition function 454 acquires an amount of change at each position within the subject when the temperature within the subject is changed by the temperature controller 5. That is, the second acquisition function 454 acquires a temperature-change distribution indicating the degree of temperature variation at each position within the three-dimensional space of the subject.

The generation function 455 generates a thermophysical properties distribution within the subject based on a plurality of tissue property distributions acquired under a plurality of states in which the distribution of temperature has been changed and the temperature-change distributions within the subject corresponding to the plurality of states. Specifically, the generation function 455 generates a thermophysical properties distribution using the temperature-change distribution at each position of the subject. More specifically, the generation function 455 generates a thermophysical properties distribution indicating an amount of change in tissue properties per a predetermined temperature change at each position of the subject. For example, the generation function 455 generates a thermophysical properties distribution between a plurality of sound speed distributions. For example, the generation function 455 generates a thermophysical properties distribution by calculating the amount of change in sound speed per 1°C temperature change at each three-dimensional position within the subject.

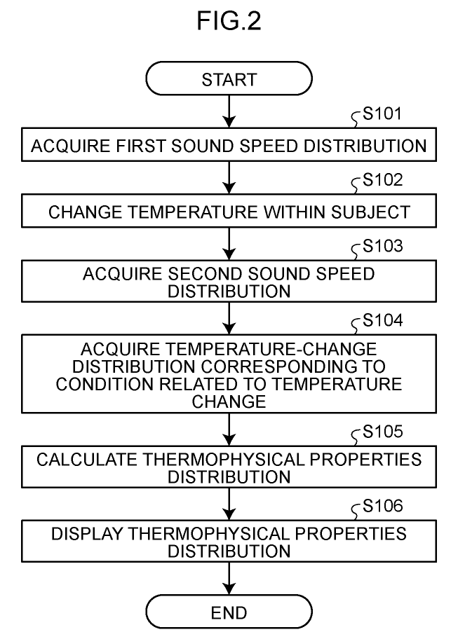

Hereinafter, the processing procedures by the ultrasonic diagnostic apparatus 10 will be described using FIG. 2, followed by a detailed explanation of each processing. FIG. 2 is a flowchart illustrating a processing procedure of the ultrasonic diagnostic apparatus 10 according to the first embodiment.

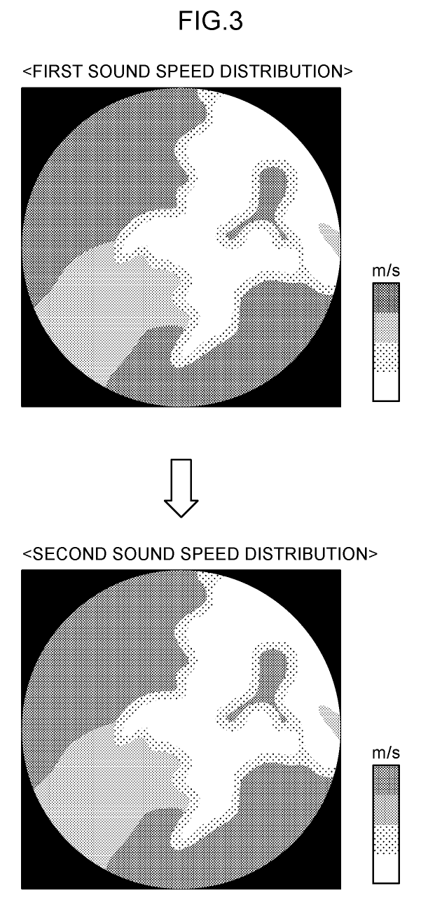

For example, as illustrated in FIG. 3, in the present embodiment, the first acquisition function 453 acquires a first sound speed distribution (Step S101). Specifically, the first acquisition function 453 acquires the first sound speed distribution based on ultrasonic data acquired by the control of the control function 451. The processing of Step S101 described above is realized by, for example, the processing circuitry 45 calling a program corresponding to the control function 451 and the first acquisition function 453 from the memory circuitry 44 and executing the program.

Next, the control function 451 changes the temperature within the subject by controlling the temperature controller 5 (Step S102). The processing of Step S102 described above is realized by, for example, the processing circuitry 45 calling a program corresponding to the control function 451 from the memory circuitry 44 and executing the program.

Next, the first acquisition function 453 acquires a second sound speed distribution (Step S103). Specifically, the first acquisition function 453 acquires the second sound speed distribution similarly as Step S101. The processing of Step S103 described above is realized by, for example, the processing circuitry 45 calling a program corresponding to the control function 451 and the first acquisition function 453 from the memory circuitry 44 and executing the program.

Next, the second acquisition function 454 acquires a temperature-change distribution corresponding to a condition related to the temperature change from the memory circuitry 44 (Step S104). The processing of Step S104 described above is realized by, for example, the processing circuitry 45 calling a program corresponding to the second acquisition function 454 from the memory circuitry 44 and executing the program.

Next, the generation function 455 calculates thermophysical property values at each position within the subject based on the first sound speed distribution, the second sound speed distribution, and the temperature-change distribution to calculate the thermophysical properties distribution (Step S105). The processing of Step S105 described above is realized by, for example, the processing circuitry 45 calling a program corresponding to the generation function 455 from the memory circuitry 44 and executing the program.

Next, the control function 451 displays the thermophysical properties distribution on the display 2 (Step S106). The processing of Step S106 described above is realized by, for example, the processing circuitry 45 calling a program corresponding to the control function 451 from the memory circuitry 44 and executing the program.

Hereinafter, a detailed explanation of each processing executed by the ultrasonic diagnostic apparatus 10 will be described.

Acquisition Processing for Sound Speed Distribution

As described in Steps S101 and S103, the first acquisition function 453 acquires the sound speed distribution indicating the sound speed at each position within the subject. Specifically, the first acquisition function 453 acquires the sound speed distribution at each three-dimensional position in a target part of the subject based on the result of the ultrasonic CT scanning controlled by the control function 451.

For example, the control function 451 transmits ultrasonic waves from a plurality of piezoelectric transducer elements on one side of a plurality of opposing piezoelectric transducer elements and receives ultrasonic waves transmitted through the target part of the subject by a plurality of piezoelectric transducer elements on the other side. The control function 451 performs such ultrasonic scanning from a plurality of directions around the target part of the subject and receives the ultrasonic waves transmitted through from each direction. The first acquisition function 453, for example, calculates the propagation time of ultrasonic waves at each point in each direction and reconstructs the sound speed distribution based on the calculated propagation times. Note that the method for acquiring the sound speed distribution is not limited to the above, but for example, it may also be acquired using AI.

Here, the acquisition of the second sound speed distribution in Step S103 may be executed immediately after the temperature controller 5 changed the temperature within the subject (immediately after Step S102), but it may be executed after a certain period of time has elapsed after the temperature controller 5 changed the temperature. For example, the thermal diffusion time constant differs for each tissue. Therefore, the timing for acquiring the second sound speed distribution may be adjusted according to which tissue is the target part (the time constant of each tissue).

FIG. 3 is a diagram for explaining an example of sound speed distribution according to the first embodiment. FIG. 3 illustrates the sound speed distribution in two dimensions, however, in practice, the first acquisition function 453 acquires the three-dimension sound speed distribution. For example, the first acquisition function 453 acquires, as illustrated in FIG. 3, the first sound speed distribution indicating the sound speed (m/s) at each position of the target part of the subject before the temperature change by the temperature controller 5, and the second sound speed distribution indicating the sound speed (m/s) at each position of the target part of the subject after the temperature change by the temperature controller 5.

Note that the second sound speed distribution may be acquired a plurality of times. That is, the first acquisition function 453 may acquire the second sound speed distribution a plurality of times at predetermined acquisition timings (for example, immediately after the temperature change, and after a certain period has elapsed following the temperature change) following the temperature change by the temperature controller 5.

### Temperature Change Processing

As described in Step S102, the temperature controller 5 changes the temperature within the subject. Specifically, the temperature controller 5 performs heating processing or cooling processing under control by the control function 451 (or control by another device). Here, the temperature controller 5 can perform temperature change processing under various conditions. For example, the temperature controller 5 can change the heating amount or heating time (or the cooling amount or cooling time).

In Step S102, the temperature controller 5 changes the temperature within the subject by performing the heating processing or the cooling processing based on preset conditions. The conditions for controlling the temperature controller 5 may be set for each tissue to be the target part, or alternatively, may be set for each subject. When conditions are set for each subject, they may be set based on subject information (height, weight, bone density, shape and size of the target part, or the like).

### Acquisition Processing of Temperature-Change Distribution

As described in Step S104, the second acquisition function 454 acquires the temperature-change distribution corresponding to the conditions related to the temperature change. Specifically, the second acquisition function 454 acquires the temperature-change distribution corresponding to the conditions including the target part, type of the temperature controller 5, the heating amount (cooling amount), the heating time (cooling time), and the subject information, or the like. Here, the temperature-change distribution according to the present embodiment includes information on temperature changes over time. That is, the temperature-change distribution includes information on the temporal progression of the amount of temperature change at each position within the target part when the temperature change processing is performed by the temperature controller 5. For example, as the temperature-change distribution in a case where the temperature controller 5 performs a heating processing for ten seconds on the target part, the progression of the amount of temperature change at each position on the target part may be obtained for a total of one minute that is ten seconds of the heating period and 50 seconds following the end of heating.

In the ultrasonic diagnostic apparatus 10 according to the first embodiment, the temperature-change distributions corresponding to the above-described conditions are pre-stored in the memory circuitry 44, and the second acquisition function 454 acquires the corresponding temperature-change distribution from among the plurality of temperature-change distributions stored in the memory circuitry 44. That is, the second acquisition function 454 acquires the condition for the temperature change processing in Step S102 and acquires the temperature-change distribution corresponding to the acquired condition from the memory circuitry 44.

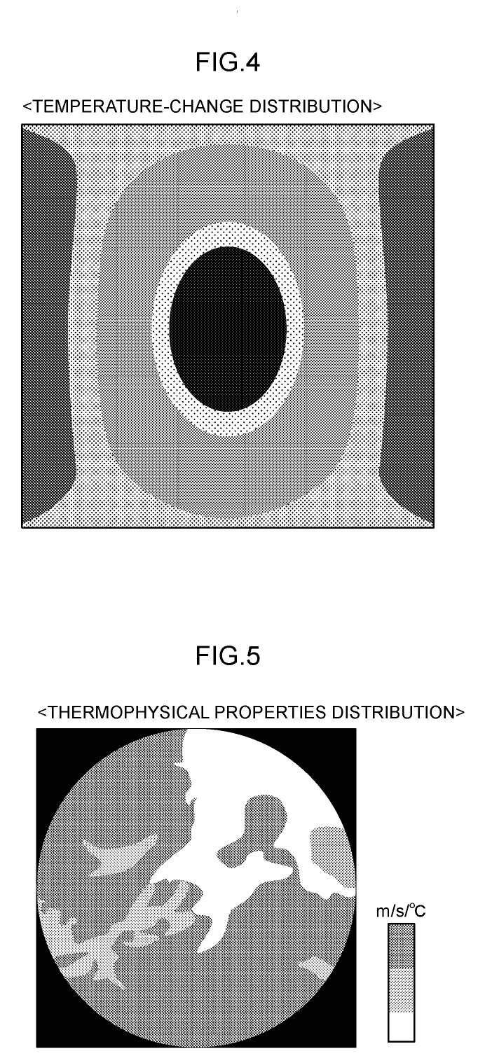

FIG. 4 is a diagram for explaining an example of temperature-change distribution according to the first embodiment. FIG. 4 illustrates the temperature-change distribution in two dimensions, however, in practice, the second acquisition function 454 acquires the three-dimensional temperature-change distribution. In addition, FIG. 4 illustrates the temperature-change distribution at one time point, however, in practice, the temperature-change distribution includes information on the temporal progression of the amount of temperature change. For example, the second acquisition function 454 acquires a temperature-change distribution indicating the degree of temperature change (how many degrees Celsius changed) at each position of the target part of the subject due to the temperature change processing performed by the temperature controller 5, as illustrated in FIG. 4.

The temperature-change distribution pre-stored in the memory circuitry 44 can be obtained using various methods.

For example, the temperature-change distribution may be obtained using a simulator that performs numerical analysis based on electromagnetic field analysis (for example, coupled numerical analysis of heat transfer based on electromagnetic field analysis) or numerical analysis based on sound field analysis (for example, coupled numerical analysis of heat transfer based on sound field analysis). In such cases, for example, by inputting the control conditions of the temperature controller 5 (for example, heating amount, heating time, etc.) and a human body model corresponding to the target part into a simulator corresponding to the type of temperature controller 5, a temperature-change distribution (simulation result) including information on the amount of temperature change over time is obtained. The memory circuitry 44 can store the simulation result for each condition.

Here, the input human body model may be deformed based on subject information. That is, in addition to the conditions described above, simulation results incorporating subject information may also be obtained. In this case, for example, the size and shape of the human body model's external form may be altered based on the body measurements (height, weight, etc.) of the subject. Alternatively, in a case where medical images of the subject have been collected, the size and shape of the internal organs within the human body model may be altered based on the collected medical images. Using a human body model tailored to the subject in this manner, the above-described simulator acquires the temperature-change distribution (simulation results) for each of the above-described conditions. The memory circuitry 44 stores simulation results for each condition, corresponding to the subject information. Specifically, the memory circuitry 44 stores a plurality of temperature-change distributions corresponding to the characteristics of each subject, and the second acquisition function 454 acquires the temperature-change distribution corresponding to the subject whose tissue property distribution has been acquired from the memory circuitry 44.

The temperature-change distribution may be acquired using actual measurements in addition to the above-described simulation. For example, by applying a temperature change to a biocompatible phantom using the temperature controller 5 and measuring the temperature of the biocompatible phantom with a temperature sensor from the start of the temperature change until the end of measurement, a temperature-change distribution including information on the amount of temperature change over time may be obtained. In this case, a temperature sensor such as a thermocouple or infrared sensor (thermography, optical fibers, etc.) may be used.

### Calculation Processing of Thermophysical Properties Distribution

As described in Step S105, the generation function 455 calculates the thermophysical properties distribution based on the first sound speed distribution, the second sound speed distribution, and the temperature-change distribution. Specifically, the generation function 455 calculates the amount of change in sound speed for each position of the target part based on the first sound speed distribution and the second sound speed distribution, and calculates the amount of change in sound speed (sound speed gradient) per a predetermined temperature change based on the calculated amount of change in sound speed and the temperature-change distribution. For example, the generation function 455 acquires the amount of temperature change corresponding to the point in time when the second sound speed distribution is acquired from the temperature-change distribution, and calculates the sound speed gradient from the amount of temperature change at each position obtained and the amount of change in sound speed at each position calculated.

Here, the generation function 455 can perform alignment between the first sound speed distribution and the second sound speed distribution, and between the sound speed distribution and the temperature-change distribution. For example, the generation function 455 performs alignment between the first sound speed distribution and the second sound speed distribution before calculating the amount of change in sound speed, and performs alignment between the first sound speed distribution (or the second sound speed distribution) and the temperature-change distribution before calculating the rate of change in sound speed. Furthermore, for the alignment method, known linear or nonlinear deformation alignment methods can be used, such as the free-form deformation (FFD) method or the large deformation diffeomorphic metric mapping (LDDMM) method may be used.

FIG. 5 is a diagram for explaining an example of a thermophysical properties distribution according to the first embodiment. FIG. 5 illustrates the thermophysical properties distribution in two dimensions, however, in practice, the generation function 455 generates the three-dimensional thermophysical properties distribution. For example, the generation function 455 generates, as illustrated in FIG. 5, the thermophysical properties distribution indicating the amount of change in sound speed per 1°C temperature change at each position of the target part.

The above example described generating a thermophysical properties distribution from two sound speed distributions, the first and second sound speed distributions, but the embodiment is not limited to this, and it may also apply when three or more sound speed distributions are acquired and a thermophysical properties distribution is generated based on them. For example, a plurality of second sound speed distributions is acquired, and the generation function 455 calculates the thermophysical properties distribution (second thermophysical properties distribution) between the plurality of second sound speed distributions based on the amount of change in sound speed between the plurality of second sound speed distributions acquired and the amount of temperature change at the corresponding time point. Furthermore, the generation function 455 may calculate the sound speed gradient at the target part of the subject by statistically processing the thermophysical properties distribution between the first sound speed distribution and the second sound speed distribution (the first thermophysical properties distribution) and the second thermophysical properties distribution using methods such as the least squares method.

In addition, as the sound speed gradient at the target part of the subject, the above-described second thermophysical properties distribution may be calculated. Furthermore, in a case where the amount of change in sound speed is small between two sound speed distributions, the amount of change may be made larger by virtually extending the time between the two sound speed distributions, and the sound speed gradient may be calculated using the amount of temperature change at the corresponding time.

### Display Processing of Thermophysical Properties Distribution

As described in Step S106, the control function 451 controls the display 2 to display the thermophysical properties distribution generated by the generation function 455. For example, the control function 451 displays an image on the display 2, assigning a pixel value corresponding to the sound speed gradient at that position to each pixel of the ultrasonic image collected by the ultrasonic CT scanning. This enables the control function 451 to display images representing differences in tissue characteristics by pixel values on the display 2.

Furthermore, the control function 451 can also display the tissue property distribution and the temperature-change distribution on the display 2. For example, the control function 451 can display an image in which each pixel of the ultrasonic image collected by the ultrasonic CT scanning is assigned a pixel value corresponding to the sound speed at that position, or an image in which each pixel is assigned a pixel value corresponding to the amount of temperature change at that position, together with the thermophysical properties distribution image on the display 2.

As described above, according to the first embodiment, the first acquisition function 453 acquires the tissue property distribution within the subject. The second acquisition function 454 acquires the temperature-change distribution within the subject in which the distribution of temperature has been changed by the temperature controller 5 that changes distribution of temperature within the subject. The generation function 455 generates a thermophysical properties distribution within the subject based on a plurality of tissue property distributions acquired under a plurality of states in which the distribution of temperature has been changed and the temperature-change distributions within the subject corresponding to the plurality of states. Therefore, the ultrasonic diagnostic apparatus 10 according to the first embodiment can generate a thermophysical properties distribution using the amount of temperature change at each location of the target part, enabling more precise imaging of thermophysical properties.

For example, even when the difference in sound speed between tissues is small, the imaging of thermophysical properties described above can reflect the differences in changes accompanying temperature change, enabling the provision of images that clearly show differences in tissue characteristics. As a result, the potential can be expanded for using imaging of thermophysical properties in diagnostics.

Furthermore, according to the first embodiment, the tissue property within the subject is the speed of sound. Therefore, the ultrasonic diagnostic apparatus 10 according to the first embodiment can image more precisely the sound speed gradient at each location within the target part.

Furthermore, according to the first embodiment, the temperature controller 5 is any one of a means for heating using ultrasonic acoustic energy, a means for heating using electromagnetic interference, a means for heating using optical interference, and a means for heating using heat transfer from a heat source. Therefore, the ultrasonic diagnostic apparatus 10 according to the first embodiment can employ various methods to change the temperature within the subject, enabling easy implementation.

Furthermore, according to the first embodiment, the second acquisition function 454 acquires the temperature-change distribution for each condition in the temperature controller 5. Therefore, the ultrasonic diagnostic apparatus 10 according to the first embodiment can acquire a temperature-change distribution corresponding to the temperature controller 5, enabling the acquisition of a more precise temperature-change distribution.

In addition, according to the first embodiment, the memory circuitry 44 stores a plurality of temperature-change distributions corresponding to the characteristics of each subject. The second acquisition function 454 acquires, from the memory circuitry 44, the temperature-change distribution corresponding to the subject whose tissue property distribution has been acquired. Therefore, the ultrasonic diagnostic apparatus 10 according to the first embodiment can generate a thermophysical properties distribution using the temperature-change distribution for each subject, enabling more precise imaging.

Furthermore, according to the first embodiment, the control function 451 displays the thermophysical properties distribution, the tissue property distribution, and the temperature-change distribution. Therefore, the ultrasonic diagnostic apparatus 10 according to the first embodiment can present appropriate information to the user.

### Second Embodiment

The first embodiment described above explains the case where the temperature-change distribution is acquired in advance and stored in the memory circuitry 44. In the second embodiment, the case where the temperature-change distribution is acquired in real time at the time of acquisition of the sound speed distribution will be described. Here, the second embodiment differs from the first embodiment in the processing performed by the second acquisition function 454. The following explanation will focus on this point.

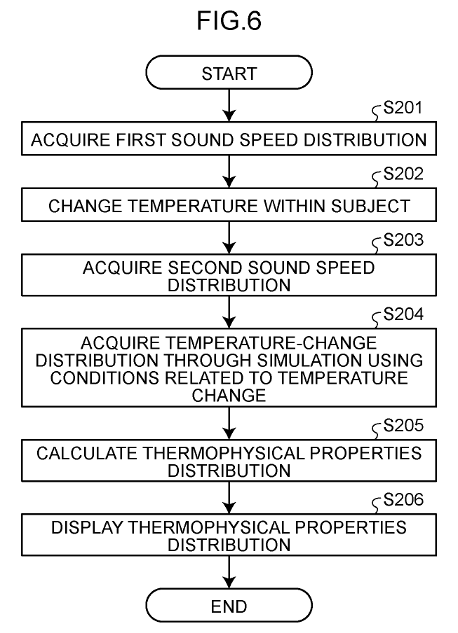

First, the processing procedures by the ultrasonic diagnostic apparatus 10 according to the second embodiment will be described using FIG. 6. FIG. 6 is a flowchart illustrating a processing procedure of the ultrasonic diagnostic apparatus 10 according to the second embodiment. Note that, Steps S201 to S203, S205, and S206 in FIG. 6 are the same processing as Steps S101 to S103, S105, and S106 in FIG. 2.

For example, as illustrated in FIG. 6, in the present embodiment, the first acquisition function 453 acquires the first sound speed distribution (Step S201), and the control function 451 changes the temperature within the subject by controlling the temperature controller 5 (Step S202).

Next, when the first acquisition function 453 acquires the second sound speed distribution (Step S203), the second acquisition function 454 acquires the temperature-change distribution through simulation using conditions related to temperature change (Step S204). The processing of Step S204 described above is realized by, for example, the processing circuitry 45 calling a program corresponding to the second acquisition function 454 from the memory circuitry 44 and executing the program.

Next, the generation function 455 calculates thermophysical property values at each position within the subject based on the first sound speed distribution, the second sound speed distribution, and the temperature-change distribution to calculate the thermophysical properties distribution (Step S205), and the control function 451 displays the thermophysical properties distribution on the display 2 (Step S206).

### Acquisition Processing of Temperature-Change Distribution

As described in Step S204, the second acquisition function 454 according to the second embodiment acquires the temperature-change distribution through simulation at the time of acquisition of the sound speed distribution. Specifically, the second acquisition function 454 acquires the temperature-change distribution within the subject through numerical analysis based on electromagnetic field analysis (for example, coupled numerical analysis of heat transfer based on electromagnetic field analysis) or numerical analysis based on sound field analysis (for example, coupled numerical analysis of heat transfer based on sound field analysis). For example, the second acquisition function 454 acquires the temperature-change distribution at the target part of the subject by executing a simulation using the type and control conditions of the temperature controller 5 and subject information.

Here, the subject information may be input at the time when the temperature-change distribution is acquired. Specifically, the input interface 3 accepts input operations for information regarding the subject, and the second acquisition function 454 acquires the temperature-change distribution within the subject using the information regarding the subject input via the input operations. Note that the information regarding the subject input via the input operation includes, for example, characteristics of the subject's anatomical structures and past medical images.

Additionally, the second acquisition function 454 can also use the results of the ultrasonic CT scanning performed in Step S201 or Step S203 for simulation. In such a case, for example, the second acquisition function 454 extracts the anatomical structures of the subject from the ultrasonic data collected by the ultrasonic CT scanning, deforms the human body model based on the extracted anatomical structures, and inputs the model into the simulation.

As described above, according to the second embodiment, the second acquisition function 454 acquires the temperature-change distribution within the subject through numerical analysis based on electromagnetic field analysis or numerical analysis based on sound field analysis. Therefore, the ultrasonic diagnostic apparatus 10 according to the second embodiment can perform imaging of thermophysical properties without requiring the prior preparation of a temperature-change distribution.

Furthermore, according to the second embodiment, the input interface 3 accepts input operations for information regarding the subject. The second acquisition function 454 acquires the temperature-change distribution within the subject using information regarding the subject input via input operations. Therefore, the ultrasonic diagnostic apparatus 10 according to the second embodiment can easily perform the acquisition processing for temperature-change distribution tailored to the subject.

### Other Embodiments

Although the above embodiment described obtaining the sound speed as a tissue property and calculating the sound speed gradient as a thermophysical property, the embodiment is not limited to this, and other physical properties (for example, CT values or NMR parameters) may also be used. In this case, the method according to the present application described above may be applied to each medical imaging diagnostic apparatus.

For example, the processing circuitry within an X-ray CT apparatus performs processing similar to the control function 451, the first acquisition function 453, the second acquisition function 454, and the generation function 455 described above. Specifically, the processing circuitry of the X-ray CT apparatus acquires CT values (CT value distribution) at each position within the target part of the subject in a plurality of states with changed temperature distributions, thereby calculating the amount of change in CT values at each position. Furthermore, the processing circuitry acquires the amount of temperature change at each position of the target part of the subject and calculates the rate of change in CT values at each position of the target part based on the amount of change in CT values and the amount of temperature change.

In addition, for example, the processing circuitry included in a magnetic resonance imaging (MRI) apparatus performs processing similar to the control function 451, the first acquisition function 453, the second acquisition function 454, and the generation function 455 described above. That is, the processing circuitry included in the MRI apparatus acquires NMR parameters (NMR parameter distribution) at each position within the target part of the subject in a plurality of states with changed temperature distributions, thereby calculating the amount of change in the NMR parameters at each position. Furthermore, the processing circuitry acquires the amount of temperature change at each position within the target part of the subject and calculates the rate of change of the NMR parameters at each position within the target part based on the amount of change in the NMR parameters and the amount of temperature change. Furthermore, the NMR parameters whose values change in accordance with temperature changes include, for example, chemical shift, spin-lattice relaxation time, and spin-spin relaxation time.

Furthermore, the above embodiments described the case where a medical imaging diagnostic apparatus executes the method according to the present application. However, the embodiments are not limited to this, and the method according to the present application may also be executed by a medical image processing apparatus.

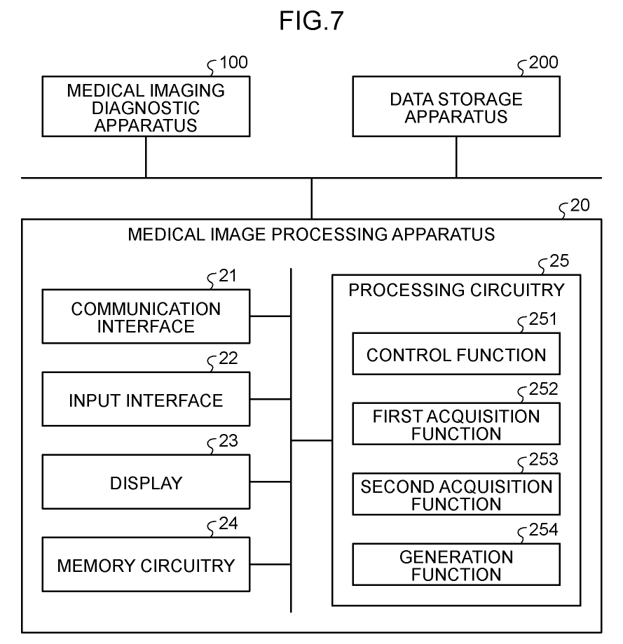

FIG. 7 is a block diagram illustrating an example of a configuration of a medical image processing apparatus according to another embodiment. For example, as illustrated in FIG. 7, a medical image processing apparatus 20 according to the present embodiment is communicably connected to a medical imaging diagnostic apparatus 100 and a data storage apparatus 200 via a network. Note that the network illustrated in FIG. 7 may also be connected to various other apparatuses and systems.

The medical imaging diagnostic apparatus 100 collects medical images by imaging the subject. Specifically, the medical imaging diagnostic apparatus 100 collects the tissue property distribution of the subject. Then, the medical imaging diagnostic apparatus 100 transmits the generated medical images and tissue property distributions to various apparatuses on the network. For example, the medical imaging diagnostic apparatus 100 includes an X-ray CT apparatus, an MRI apparatus, an ultrasonic diagnostic apparatus, and the like.

The data storage apparatus 200 stores various medical images and tissue property distributions regarding the subject. Specifically, the data storage apparatus 200 receives medical images and tissue property distributions from the medical imaging diagnostic apparatus 100 via the network, and stores these medical images and tissue property distributions in its internal memory circuitry. For example, the data storage apparatus 200 is implemented by computer equipment such as servers or workstations.

The medical image processing apparatus 20 generates a thermophysical properties distribution from the tissue property distribution and temperature-change distribution of the subject. For example, the medical image processing apparatus 20 is installed in medical facilities such as hospitals and clinics, supporting various diagnoses and treatment planning conducted by users such as physicians. In other words, the medical image processing apparatus 20 may be utilized to generate thermophysical properties distributions at the user's desired timing after a plurality of tissue property distributions has been collected by the medical imaging diagnostic apparatus 100. For example, the medical image processing apparatus 20 is implemented by computer equipment such as servers or workstations.

For example, the medical image processing apparatus 20 includes a communication interface 21, an input interface 22, a display 23, memory circuitry 24, and processing circuitry 25.

The communication interface 21 controls the transmission and communication of various data sent and received between the medical image processing apparatus 20 and other apparatuses connected via a network. Specifically, the communication interface 21 is connected to the processing circuitry 25, transmitting data received from other apparatuses to the processing circuitry 25, or transmitting data received from the processing circuitry 25 to other apparatuses. For example, the communication interface 21 is implemented by a network card, a network adapter, a network interface controller (NIC), or the like.

The input interface 22 accepts input operations from the user for various instructions and various types of information. Specifically, the input interface 22 is connected to the processing circuitry 25, converting input operations received from the user into electrical signals and transmitting the signals to the processing circuitry 25. For example, the input interface 22 may be implemented by a trackball, switch buttons, a mouse, a keyboard, a touchpad that performs input operations by touching the operating surface, a touchscreen integrating a display screen and a touchpad, a non-contact input interface using an optical sensor, and a voice input interface, and the like. Note that in the present specification, the input interface 22 is not limited to the one having a physical operation part such as a mouse, keyboard, etc. For example, examples of the input interface 22 include a processing circuit of an electrical signal that receives an electrical signal corresponding to an input operation from an external input device mounted separately from the device and transmits the electrical signal to the control circuit.

The display 23 displays various types of information and various types of data. Specifically, the display 23 is connected to the processing circuitry 25 and displays various types of information and various types of data that are received from the processing circuitry 25. For example, the display 23 is implemented using an LCD display, a cathode ray tube (CRT) display, a touch panel, or the like.

The memory circuitry 24 stores various types of data and various programs. Specifically, the memory circuitry 24 is connected to the processing circuitry 25, storing data received from the processing circuitry 25 or reading the stored data and transmitting the data to the processing circuitry 25. For example, the memory circuitry 24 is implemented using semiconductor memory devices such as a random access memory (RAM) or a flash memory, or by a hard disk, an optical disk, etc.

The processing circuitry 25 controls the entire medical image processing apparatus 20. For example, the processing circuitry 25 performs various processing operations in response to input operations received from the user via the input interface 22. For example, the processing circuitry 25 receives data transmitted by other apparatuses via the communication interface 21 and stores the received data in the memory circuitry 24. Additionally, for example, the processing circuitry 25 transmits data received from the memory circuitry 24 to the communication interface 21, thereby sending that data to other apparatuses. Additionally, for example, the processing circuitry 25 displays the data received from the memory circuitry 24 on the display 23.

As illustrated in FIG. 7, the processing circuitry 25 of the medical image processing apparatus 20 executes a control function 251, a first acquisition function 252, a second acquisition function 253, and a generation function 254. The processing circuitry 25 described above is implemented by a processor, for example.

The control function 251 executes processing similar to the control function 451 described above. Specifically, the control function 251 executes display control to display the thermophysical properties distribution, the tissue property distribution, and the temperature-change distribution on the display 23. Note that the control function 251 does not necessarily control a temperature controller (not illustrated).

The first acquisition function 252 acquires a distribution of tissue properties of the subject. Specifically, the first acquisition function 252 acquires the tissue property distribution of the subject from the medical imaging diagnostic apparatus 100 or the data storage apparatus 200 via a network. For example, the first acquisition function 252 acquires the tissue property distribution corresponding to the subject information input via the input interface. Additionally, the first acquisition function 252 can also acquire the sound speed distribution by performing reconstruction using the results of the ultrasonic CT scanning stored by the medical imaging diagnostic apparatus 100 or the data storage apparatus 200.

The second acquisition function 253 executes processing similar to the second acquisition function 454 described above. Specifically, the second acquisition function 253 acquires conditions related to the temperature change of the subject and acquires the temperature-change distribution of the subject corresponding to the acquired conditions. For example, the second acquisition function 253 acquires the temperature-change distribution of the subject corresponding to the conditions from among the temperature-change distributions stored in advance in the data storage apparatus 200 or the memory circuitry 24. Alternatively, the second acquisition function 253 acquires the temperature-change distribution of the subject through simulation using conditions related to the subject's temperature change.

The generation function 254 executes processing similar to the generation function 455 described above. Specifically, the generation function 254 generates a thermophysical properties distribution from the tissue property distribution acquired by the first acquisition function 252 and the temperature-change distribution acquired by the second acquisition function 253.

Note that the term "processor" used in the above description refers to, for example, a central processing unit (CPU), a graphics processing unit (GPU), or an application specific integrated circuit (ASIC), a programmable logic device (for example, a simple programmable logic device (SPLD), a complex programmable logic device (CPLD), and a field programmable gate array (FPGA)). The processor reads and executes programs stored in a memory to perform its functions. Alternatively, instead of storing the program in the memory, it is acceptable to configure the processor to embed the program directly within its circuit. In this case, the processor achieves its function by reading and executing the program embedded within the circuit. Furthermore, the processors in the present embodiment are not necessarily configured as a single circuit per processor, but they may also be configured by combining a plurality of independent circuits into a single processor to achieve their functions.

In addition, constituent components of each apparatus illustrated in the above embodiments are functionally conceptual and are not necessarily physically configured as illustrated in the drawings. That is, the specific aspects of distribution and integration of the apparatuses are not limited to those illustrated in the diagrams, and all or some of the apparatuses may be distributed or integrated functionally or physically in desired units depending on various kinds of loads, states of use, and the like. Further, all or some of the processing functions performed by each apparatus can be realized by a CPU and a program analyzed and executed by the CPU or realized as hardware with wired logic

Furthermore, the method described in the above-mentioned embodiments can be implemented by executing a pre-prepared program on a computer such as a personal computer or workstation. This program can be distributed via networks such as the Internet. Additionally, this program may be recorded on non-volatile storage media readable by a computer, such as hard disks, flexible disks (FD), CD-ROMs, MO, DVD, and Flash Memory such as USB memory and SD card memory, and may be executed by being read from such non-volatile storage media by a computer.

As described above, according to the embodiment, it is possible to perform more precise imaging of thermophysical properties.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising processing circuitry configured to:
acquire a tissue property distribution within a subject;
change a temperature distribution within the subject;
acquire a temperature-change distribution within the subject in which the temperature distribution has been changed; and
generate a thermophysical properties distribution within the subject based on a plurality of tissue property distributions acquired under a plurality of states in which the temperature distribution has been changed and the temperature-change distributions within the subject corresponding to the plurality of states.

2. A medical image processing apparatus comprising processing circuitry configured to:
acquire a tissue property distribution within a subject;
acquire a temperature-change distribution within the subject in which a temperature distribution has been changed by a temperature controller that changes the temperature distribution within the subject; and
generate a thermophysical properties distribution within the subject based on a plurality of tissue property distributions acquired under a plurality of states in which the temperature distribution has been changed and the temperature-change distributions within the subject corresponding to the plurality of states.

3. The medical image processing apparatus according to claim 2, wherein the processing circuitry is configured to generate a thermophysical properties distribution using the temperature-change distribution at each position of the subject.

4. The medical image processing apparatus according to claim 2, wherein the processing circuitry is configured to acquire sound speed distribution indicating sound speed at each position within the subject.

5. The medical image processing apparatus according to claim 4, wherein the processing circuitry is configured to generate a thermophysical properties distribution between a plurality of sound speed distributions.

6. The medical image processing apparatus according to claim 2, wherein the tissue property within the subject is any one of sound speed, a computed tomography (CT) value, and a nuclear magnetic resonance (NMR) parameter.

7. The medical image processing apparatus according to claim 2, wherein the temperature controller is configured to change the temperature distribution within the subject using any one of methods including a method of heating by ultrasonic acoustic energy, a method of heating by electromagnetic interference, a method of heating by optical interference, and a method of heating by heat transfer from a heat source.

8. The medical image processing apparatus according to claim 2, wherein the processing circuitry is configured to acquire the temperature-change distribution within the subject through numerical analysis based on electromagnetic field analysis or numerical analysis based on sound field analysis.

9. The medical image processing apparatus according to claim 8, wherein the processing circuitry is configured to acquire the temperature-change distribution for each condition in the temperature controller.

10. The medical image processing apparatus according to claim 2, further comprising memory circuitry storing a plurality of temperature-change distributions corresponding to characteristics of each subject, wherein
the processing circuitry is configured to acquire the temperature-change distribution corresponding to the subject whose tissue property distribution has been acquired from the memory circuitry.

11. The medical image processing apparatus according to claim 8, wherein the processing circuitry is configured to:
accept input operations for information regarding the subject; and
acquire the temperature-change distribution within the subject using information regarding the subject input via the input operations.

12. The medical image processing apparatus according to claim 2, wherein the processing circuitry is configured to display at least one of the thermophysical properties distribution, the tissue property distribution, and the temperature-change distribution.

13. A method comprising:
acquiring a tissue property distribution within a subject;
acquiring a temperature-change distribution within the subject in which a temperature distribution has been changed by a temperature controller that changes the temperature distribution within the subject; and
generating a thermophysical properties distribution within the subject based on a plurality of distributions of tissue properties acquired in a plurality of states in which the temperature distribution has been changed and the temperature-change distribution within the subject corresponding to the plurality of states.
